(19) United States
(12) Patent Application Publication
(10) Pub. No.: US 2025/0277267 A1
(43) Pub. Date: Sep. 4, 2025

(54) TECHNIQUE FOR DIAGNOSING AND MONITORING NEURODEGENERATIVE DISEASES ON BASIS OF BODY FLUID ANALYSIS

(71) Applicant: KOREA RESEARCH INSTITUTE OF BIOSCIENCE AND BIOTECHNOLOGY, Daejeon (KR)

(72) Inventors: Eun Kyung Lim, Daejeon (KR); Jae Woo Lim, Daejeon (KR); Min Ho Moon, Daejeon (KR); Su Jin Kim, Daejeon (KR); Byung Hoon Kang, Daejeon (KR); Tae Joon Kang, Daejeon (KR); Seung Beom Seo, Daejeon (KR); Seong Uk Son, Daejeon (KR); Kyu Sun Lee, Daejeon (KR); Ju Yeon Jung, Daejeon (KR)

(21) Appl. No.: 18/278,584

(22) PCT Filed: Feb. 17, 2022
(86) PCT No.: PCT/KR2022/002357

§ 371 Date: Aug. 23, 2023

(30) Foreign Application Priority Data
Feb. 24, 2021 (KR) 10-2021-0024490

(51) Int. Cl. C12Q 1/6883; B01L 3/00

(52) U.S. Cl. CPC C12Q 1/6883; B01L 3/502715; B01L 2200/027; B01L 2200/0647; B01L 2300/069; C12Q 2600/178

(57) ABSTRACT

The present invention relates to a technique for diagnosing and monitoring neurodegenerative diseases on the basis of body fluid analysis. If a detection system of the present invention is used, effective real-time diagnostic efficiency can be exhibited while a problem such as noise is minimized. Particularly, since miRNAs present in trace amounts are detected in a high detection efficiency and are diagnosed, an excellent diagnostic effect on neurodegenerative diseases including Alzheimer's disease is exhibited.

### CROSS-REFERENCE TO RELATED APPLICATIONS

This is a U.S. National Phase of International Application No. PCT/KR2022/002357, filed Feb. 17, 2022, which claims priority to Korean Patent Application No. 10-2021-0024490, filed Feb. 24, 2021, the disclosures of which are hereby incorporated herein by reference.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “59386_SeqListing.txt”, which was created on Feb. 21, 2024, and is 7,749 bytes in size. The subject matter of the Sequence Listing is incorporated herein in its entirety by reference.

### TECHNICAL FIELD

The present invention relates to a technique for diagnosing and monitoring neurodegenerative diseases on the basis of body fluid analysis.

### BACKGROUND ART

Molecular diagnosis is a diagnostic method that detects or analyzes nucleic acids such as DNAs or RNAs, and has the advantage of being very accurate and obtaining a lot of information compared to other diagnostic methods since the specificity of base sequences is used. In addition, molecular diagnosis is a field with a large market size and a fast growth rate due to a very wide range of applications, such as cancer diagnosis, diagnosis of human or livestock infectious diseases, pathogen antibiotic resistance test, food test, blood test, genetic test, and the like. In particular, on-site molecular diagnosis is the most active research field because the molecular diagnosis is able to expand the area of molecular diagnosis through strengthening access to medical support, immediate analysis and prescription of results, reduction in the number of hospital visits and waiting time, and the like.

In particular, a method of labeling and detecting nucleic acids that are difficult to be detected in natural state thereof has been applied to various fields of molecular biology or cell biology. Nucleic acids with labeled substances attached have been widely used in order to detect signals on southern blotting, northern blotting, in situ hybridization, and nucleic acid microarrays using specific hybridization reactions. A method of amplifying DNA while simultaneously labeling DNA using labeled monomers (labeled dNTPs) or labeled primers in a polymerase chain reaction (PCR) is known. The thus labeled DNA is able to be detected with a microarray.

The method of labeling nucleic acids while simultaneously performing PCR has an advantage of not requiring a separate step for labeling, but has a disadvantage in that when a monomer labeled with a fluorescent dye or the like is used, PCR efficiency is lower than that of using an unlabeled monomer. In addition, since RNA is not able to be amplified by PCR, detecting RNA by PCR labeling requires a step of preparing cDNA through reverse transcription, and in particular, short RNAs such as microRNAs (miRNAs) have a problem in that cDNA preparation is cumbersome. Accordingly, there is an urgent need to develop a nucleic acid detection technology having more improved sensitivity and specificity.

The methods described above are easy to detect a nucleic acid to be targeted when a large amount of detection nucleic acids are present. Although these methods are still widely used, it becomes very challenging to detect target nucleic acids in low quantities (low sensitivity). Moreover, there are frequent cases of erroneous detection of non-specific targets while failing to detect specific targets due to various interfering factors (low specificity).

Meanwhile, catalytic hairpin assembly, which is an isothermal and non-enzyme-free signal amplification reaction, is a reaction that produces a large amount of double-stranded products in which two types of metastable hairpin probes are bound by acting a single-stranded nucleic acid as a catalyst to repeatedly perform strand displacement reactions on the two types of hairpin probes, which has been used in the development of detection technologies for various biomaterials.

Neurodegenerative diseases are difficult to make a definitive diagnosis, and moreover, primary care settings have difficulty in diagnosis since diagnostic evaluation is often performed mainly depending on clinical features. In addition, structural and functional brain images have been attempted for objective diagnosis of dementia, but roles thereof are mainly at the level of being used as a means to exclude other dementias, and are not diagnostic methods for definitive diagnosis since neuroimaging abnormal findings are possible even in normal elderly.

In other words, the most reliable diagnosis method to date is to identify biomarkers (amyloid beta & phosphorylated tau protein) in the brain (postmortem) of Alzheimer's patients obtained through autopsy after death, which is closer to a diagnostic marker for late-stage dementia rather than a diagnostic marker for early/middle-stage dementia. Accordingly, the current cerebrospinal fluid test is merely a test method that mainly targets amyloid beta peptide as a standard for body fluid test.

Under this background, it is necessary to conduct research on improvement of cerebrospinal fluid test technology for diagnosing neurodegenerative diseases, standardization thereof, as well as the discovery of diagnostic biomarkers present in body fluids, and development of highly sensitive detection technology capable of detecting the markers.

### DISCLOSURE

### Technical Problem

The present inventors have made earnest efforts to develop a rapid and accurate method for diagnosing neurodegenerative diseases, and thus manufactured a system in which two types of probes are placed in different liposomes and released to perform a reaction after when the liposomes are degraded by a buffer solution (reaction buffer) containing a surfactant. This system has the advantage of easily detecting a small amount of miRNA, etc., by minimizing problems such as noise caused by interference between probes. Accordingly, the present inventors confirmed that the present invention effectively showed the efficiency of diagnosing neurodegenerative diseases, and completed the present invention.

An object of the present invention is to provide a composition containing a hydrogel for detecting miRNAs, wherein the hydrogel comprises a liposome comprising a first probe having a hairpin structure in which a reporter is conjugated to the 5′ end and a quencher is conjugated to the 3′ end and having a sequence complementary to a target miRNA; and a liposome comprising a second probe having a hairpin structure and having a sequence complementary to the first probe.

Another object of the present invention is to provide a composition containing a hydrogel for diagnosing neurodegenerative diseases, wherein the hydrogel comprises a liposome comprising a first probe having a hairpin structure in which a reporter is conjugated to the 5′ end and a quencher is conjugated to the 3′ end and having a sequence complementary to a target miRNA; and a liposome comprising a second probe having a hairpin structure and having a sequence complementary to the first probe.

### Technical Solution

In one general aspect, the present invention provides a composition containing a hydrogel for detecting miRNAs, wherein the hydrogel comprises a liposome comprising a first probe having a hairpin structure in which a reporter is conjugated to the 5′ end and a quencher is conjugated to the 3′ end and having a sequence complementary to a target miRNA; and a liposome comprising a second probe having a hairpin structure and having a sequence complementary to the first probe.

In the present invention, “hydrogel” is a concept encompassing a gel containing water as a basic component, a gel containing water as a dispersion medium, or a hydrophilic gel.

Hydrogel particles may comprise a hydrophilic monomer or polymer. In another aspect of the present invention, particles of the hydrogel may contain at least one selected from the group consisting of natural polymer, acrylic monomer or polymer, polyacrylamide-based monomer or polymer, phosphatidyl choline, hyaluronic acid-based monomer or polymer, carboxymethyl cellulose, alginate, chitosan, poly(e-caprolactone), poly(lactic acid), poly(glycolic acid), polyethylene glycol, hydroxyapatite, tricalcium phosphate, and a mixture thereof.

The natural polymer comprises at least one selected from the group consisting of polysaccharides derived from red algae, such as carrageenan, agar, and agarose; polysaccharides containing mannose, such as mannan, galactomannan, glucomannan and derivatives thereof; and natural gums such as locust bean gum, guar gum, xanthan gum, gum arabic, gellan gum, and gum karaya.

The acrylic monomer or polymer comprises hydrophilic acrylic monomers or polymers, specifically at least one selected from the group consisting of polyethylene glycol diacrylate, polyethylene glycol methacrylate, polymethylmethacrylate (PMMA), hydroxyethyl acrylate (HEA), and hydroxyethyl methacrylate (HEMA).

In still another aspect of the present invention, the hydrogel particle may preferably comprise an acrylic monomer or polymer capable of radical polymerization in order to secure wide usability, and specifically, may preferably comprise a polyethylene glycol acrylate-based monomer or polymer.

More preferably, polyethylene glycol and a polyacrylamide-based monomer or polymer may be mixed and used. More specifically, the hydrogel particle may comprise at least one selected from the group consisting of polyethylene glycol; and polyethylene glycol diacrylate, polyethylene glycol methacrylate, polymethylmethacrylate (PMMA), hydroxyethyl acrylate (HEA), and hydroxyethyl methacrylate (HEMA), and more specifically, polyethylene glycol and polyethylene glycol diacrylate may be mixed and used. In other words, the hydrogel particle may comprise a mixture of polyethylene glycol and polyethylene glycol diacrylate.

A mixing ratio of polyethylene glycol and polyethylene glycol diacrylate is preferably a weight ratio of 1:0.5 to 2, and more specifically, approximately 1:1. Even more preferably, in a hydrophilic aqueous solution, polyethylene glycol and polyethylene glycol diacrylate may be mixed in a weight ratio of about 3:0.5 to 2:0.5 to 2 (hydrophilic aqueous solution:polyethylene glycol polyethylene glycol diacrylate), and more preferably 3:11.

The polymer capable of constituting the hydrogel is preferably a photocurable type, and more preferably photocurable by ultraviolet irradiation. In other words, the hydrogel may be produced by photocuring.

A photoinitiator may initiate free radical polymerization and/or crosslinking with the use of light. Examples of suitable photoinitiators comprise, but are not limited to, benzoin methyl ether, diethoxyacetophenone, benzoylphosphine oxide, 2-hydroxy-2-methyl propiophenone (HMPP), 1-hydroxycyclohexyl phenyl ketone, and Darocur (brand name) and Irgacure (brand name) types, preferably Darocur 1173 and 2959. Examples of benzoylphosphine initiators comprise 2,4,6-trimethylbenzoyl diphenylphosphine oxide; bis-(2,6-dichlorobenzoyl)-4-N-propylphenylphosphine oxide; and bis-(2,6-dichlorobenzoyl)-4-N-butylphenylphosphine oxide. For example, reactive photoinitiators capable of being incorporated into macromers, or capable of being used as specific monomers, are also suitable.

If a photoinitiator is contained, polymerization may be initiated by actinic radiation, for example by specific ultraviolet light having a suitable wavelength. Spectral requirements may be controlled, if appropriate, by the addition of suitable photosensitizers.

The hydrogel has a property of supporting liposomes through a porous structure with open pores inside. In addition, the porous structure of the hydrogel is advantageous for foreign substances (miRNA for diagnosis) to flow into the interior through diffusion, and the multifaceted three-dimensional structure inside the hydrogel has an advantage in that the liposome is able to be immobilized inside the hydrogel without chemical binding.

As used herein, the liposome is a spherical vesicle structure formed of one or more artificially made lipid bilayers.

The lipid forming the liposome of the present invention is not particularly limited and may be a known lipid. Examples of the lipid may comprise phospholipids, glycolipids, sterols, cationic lipids, and the like, polyglycerol alkyl ethers, polyoxyethylene alkyl ethers, alkyl glycosides, alkylmethyl glucamides, alkyl sucrose esters, dialkyl polyoxyethylene ether, dialkyl polyglycerol ether, and the like, amphiphilic block copolymers such as polyoxyethylene-polylactic acid, and the like, long-chain alkylamines or long-chain fatty acid hydrazides, and the like.

For example, the lipid is preferably at least one selected from the group consisting of natural or synthetic phospholipids such as phosphatidylcholine (such as soybean phosphatidylcholine, egg yolk phosphatidylcholine, bovine phosphatidylcholine, dilauroylphosphatidylcholine, dimyristoylphosphatidylcholine, dipalmitoylphosphatidylcholine distearoylphosphatidylcholine), phosphatidylethanolamine (dilauroylphosphatidylethanolamine, dimyristoylphosphatidylethanolamine, dipalmitoylphosphatidylethanolamine or distearoylphosphatidylethanolamine, dioleoylphosphatidylethanolamine), phosphatidylserine (such as dilauroylphosphatidylserine, dimyristoylphosphatidylserine, dipalmitoylphosphatidylserine or distearoylphosphatidylserine), phosphatidic acid, phosphatidylglycerol (such as dilauroylphosphatidylglycerol, dimyristoylphosphatidylglycerol, dipalmitoylphosphatidylglycerol or distearoylphosphatidylglycerol), phosphatidylinositol (such as dilauroylphosphatidylinositol, dimyristoylphosphatidylinositol, dipalmitoylphosphatidylinositol or distearoylphosphatidylinositol), rhizophosphatidylcholine, sphingomyelin, egg yolk lecithin, soybean lecithin or hydrogenated phospholipid.

Examples of the glycolipid may comprise glyceroglycolipid, sphingoglycolipid, and the like. Examples of the glyceroglycolipid may comprise digalactosyldiglycerides (such as digalactosyldilauroyl glyceride, digalactosyldimyristoylglyceride, digalactosyldipalmitoylglyceride or digalactosyldistearoylglyceride) or galactosyldiglycerides (such as galactosyl dilauroyl glyceride, galactosyl dimyristoyl glyceride, galactosyl dipalmitoyl glyceride or galactosyl distearoyl glyceride), and the like. Examples of the sphingoglycolipid may comprise galactosyl cerebroside, lactosyl cerebroside, ganglioside, and the like.

The sterol may be cholesterol, cholesterol hexasuccinate, 3β-[N—(N′,N′-dimethylaminoethane)carbamoyl]cholesterol, ergosterol, lanosterol, or the like.

The cationic lipid may comprise dioctadecylamidoglycylspermidine (DOGS), dimethyldioctadecylammonium bromide (DDAB), L-a-dioleoyl phostatidylethanolamine (DOPE), [N—(N,N′-dimethylaminoethane) carbamoyl]cholesterol (DC-Chol), N-[1-(2,3-dioleyloxy)propyl]-N,N,N-trimethylammonium bromide (DOTMA), 2,3-dioleoyloxy-N-[2-(sperminecarboxamido-O-ethyl]-N,N-dimethyl-propanaminium trifluoroacetate (DOSPA), 1-[2-(oleoyloxy)-ethyl]-2-oleyl-3-(2-hydroxyethyl)imidazolinium chloride (DOTIM), 1,2-dimyristyloxypropyl-3-dimethyl-hydroxy ethyl ammonium bromide (DMRIE), 1,2-dimyristoyl-3-dimethylammonium propane (DMDAP), 1,2-dipalmitoyl-3-dimethylammonium propane (DPDAP), 1,2-dilauroyl-3-dimethylammonium propane (DLDAP), 1,2-distearoyl-3-dimethylammonium propane (DSDAP), 1,2-dioleoyl-3-dimethylammonium propane (DODAP), 1,2-dimyristyl-3-dimethylammonium propane (DMDAP), 1,2-dipalmityl-3-dimethylammonium propane (DPDAP), 1,2-dilauryl-3-dimethylammonium propane (DLDAP), 1,2-distearyl-3-dimethylammonium propane (DSDAP), 1,2-dioleyl-3-dimethylammonium propane (DODAP), 1,2-dimyristoyl-3-trimethylammonium propane (DMTAP), 1,2-dipalmitoyl-3-trimethylammonium propane (DPTAP), 1,2-dilauroyl-3-trimethylammonium propane (DLTAP), 1,2-distearoyl-3-trimethylammonium propane (DSTAP), dioleoyl-3-trimethylammonium propane (DOTAP), 1,2-dimyristyl-3-trimethylammonium propane (DMTAP), 1,2-dipalmityl-3-trimethylammonium propane (DPTAP), 1,2-dilauryl-3-trimethylammonium propane (DLTAP), 1,2-distearyl-3-trimethylammonium propane (DSTAP), 1,2-dioleyl-3-trimethylammonium propane (DOTAP), and the like.

The liposome-forming lipid may be used alone or in combination of two or more.

According to an embodiment of the present invention, the liposome may be prepared using a conventional production process. For example, a lipid membrane hydration method may be used. This method is to form a liposome by hydrating a lipid membrane, and any solution for hydrating the lipid membrane may be used without limitation as long as it is able to hydrate the lipid membrane.

According to an embodiment of the present invention, the liposome may be prepared by mixing phosphatidylcholine (PC), dioleoyl-3-trimethylammonium propane (DOTAP) and cholesterol (5-cholesten-3β-ol). It is preferable to mix and use phosphatidylcholine (PC), cholesterol (5-cholesten-3β-ol), and dioleoyl-3-trimethylammonium propane (DOTAP) in a molar ratio (PC:cholesterol:DOTAP) of about 1:0.2 to 0.8:0.05 to 0.2, preferably 1:0.5:0.1.

These liposomes may be used in any form as long as they are spherical vesicles formed of a lipid bilayer capable of supporting probes. Preferably, cationic liposomes may be used.

The liposome according to the present invention may be degraded by a surfactant, and thus the probe supported thereon may be released into the hydrogel.

Examples of the surfactant may comprise, but are not limited to, cetyl trimethylammonium bromide, hexadecyl trimethyl ammonium bromide, dodecyl betaine, dodecyl dimethylamine oxide, 3-(N, N-dimethylpalmitylammonio) propane sulfonate), Tween 20, Tween 80, Triton-X-100, polyethylene glycol monooleyl ether, triethylene glycol monododecyl ether, octyl glucoside, N-nonanoyl-N-methylglucamine, and the like.

According to an embodiment of the present invention, the surfactant may be Triton X-100.

The surfactant may be contained in a buffer solution in an amount of about 0.5% to 5% by weight, preferably 0.6% to 2% by weight, and more preferably about 1% by weight.

The liposome according to the present invention may support the first probe, wherein the first probe has a hairpin structure in which a reporter is conjugated to the 5′ end and a quencher is conjugated to the 3′ end and has a sequence complementary to a target miRNA″, or may support the second probe, wherein the second probe has a hairpin structure and has a sequence complementary to the first probe.

In other words, by supporting the first probe and the second probe on separate liposomes, it is possible to prevent each of the probes from mixing prior to the reaction, thereby removing possible noise occurring therefrom.

The probe of the present invention has a hairpin structure. The hairpin structure may be naturally occurring or may be artificially introduced. For example, the detection probe may have a hairpin structure formed by adding two complementary oligonucleotide sequences to the two ends of the detection probe. In such embodiments, the two complementary oligonucleotide sequences form an arm (stem) having a hairpin structure. The arm having a hairpin structure may have any desired length, for example, 2-15 nt, for example, 3-7 nt, 4-9 nt, 5-10 nt, or 6-12 nt.

The first probe has a hairpin structure in which a reporter is conjugated to the 5′ end and a quencher is conjugated to the 3′ end and has a sequence complementary to a target miRNA. The first probe corresponds to the detection probe.

The reporter group conjugated to the 5′ end thereof may independently have a fluorescent group. For example, the reporter group may have the fluorescent group such as ALEX-350, FAM, VIC, TET, CAL Fluor® Gold 540, JOE, HEX, CAL Fluor Orange 560, TAMRA, CAL Fluor Red 590, ROX, CAL Fluor Red 610, TEXAS RED, CAL Fluor Red 635, Quasar 670, CY3, CY5, CY5.5, or Quasar 705.

The quencher group conjugated to the 3′ end thereof is a molecule or group capable of absorbing/quenching fluorescence. For example, groups such as DABCYL, BHQ (for example, BHQ-1 or BHQ-2), ECLIPSE, and/or TAMRA may be used.

The second probe may have a hairpin structure and have a sequence complementary to the first probe.

Term ‘hybridization’ of the present invention means that complementary single-stranded nucleic acids form a double-stranded nucleic acid. The hybridization may occur when complementarity between two nucleic acid strands is perfect (perfect match) or even when some mismatch bases are present. The degree of complementarity required for hybridization may vary depending on hybridization conditions, particularly temperature.

These first and second probes are used for catalytic hairpin assembly (CHA). This corresponds to a reaction in which a single-stranded nucleic acid acts as a catalyst to repeatedly perform strand displacement reactions on two types of metastable hairpin probes, thereby producing a large amount of double-stranded products in which the two types of hairpin probes are bound. As described above, the first probe is modified with a fluorophore (FAM) and a quencher (BHQ1), respectively, and the target mRNA initiates fluorescence recovery and catalyzes the assembly of the first probe and the second probe through the toehold-mediated hairpin DNA circuit (ii).

The above reaction may be used to solve the low sensitivity problem of the existing technology. In addition, through this, the reaction may be performed without an additional temperature change and temperature control device, the addition of enzymes or other substrates is unnecessary, and the reaction may proceed without requiring complicated and time-consuming experimental procedures.

In other words, the hydrogel comprises a liposome comprising a first probe having a hairpin structure in which a reporter is conjugated to the 5′ end and a quencher is conjugated to the 3′ end and having a sequence complementary to a target miRNA; and a liposome comprising a second probe having a hairpin structure and having a sequence complementary to the first probe and preserves the probes by the liposomes having the first and second probes supported thereon, respectively, to remove noise caused by their mutual interference. In addition, the probes are released from the liposomes when membranes of the liposomes are broken due to a surfactant or the like, and then react with the target miRNAs.

A composition containing a hydrogel for miRNA detection according to the present invention specifically binds to miRNAs and provides information on the presence or absence of miRNAs through the CHA reaction.

MicroRNAs (miRNAs) are small single-stranded non-coding RNA molecules containing about 22 nucleotides and found in plants, animals, and viruses. In other words, “miRNAs (microRNAs)” are short non-coding RNAs capable of regulating gene expression at the transcription and translation level. While being conserved throughout evolution, miRNAs are involved in fundamental biological processes such as cell cycle, differentiation, development, metabolism, patterning and aging.

MicroRNAs and target genes regulated thereby may play an important role in predicting the mechanisms of various diseases. Therefore, miRNAs are recognized as biomarkers capable of being used for diagnosis, prediction, and prognosis of diseases by indicating increased or decreased patterns of abnormal miRNA expression according to various diseases such as cancer, degenerative diseases, diabetes, and cardiovascular diseases. Meanwhile, miRNAs exist in trace amounts in biological materials, and selective and highly sensitive assays are required to detect these miRNAs.

A method for detecting miRNAs according to the present invention is characterized in that the first probe and the second probe are separated and preserved by the liposomes having the first probe and the second probe supported thereon, respectively, to remove noise caused by mutual interference thereof and to greatly amplify the signal through the CHA reaction, thereby making it possible to detect miRNAs present in trace amounts with high sensitivity. The trace amount refers to a small amount such as a nanomolar (nM) or picomolar (pM) in a sample.

Any miRNA to be detected in the present invention may be included in the detection target in the present invention.

The present invention provides a composition containing a hydrogel for diagnosing neurodegenerative diseases, wherein the hydrogel comprises a liposome comprising a first probe having a hairpin structure in which a reporter is conjugated to the 5′ end and a quencher is conjugated to the 3′ end and having a sequence complementary to a target miRNA; and a liposome comprising a second probe having a hairpin structure and having a sequence complementary to the first probe.

The term “neurodegenerative diseases” may be any one selected from the group consisting of Parkinson's disease, Huntington's disease, Alzheimer's disease, mild cognitive impairment, senile dementia, diabetic dementia, alcoholic dementia, vascular dementia, amyotrophic lateral sclerosis, spinocerebellar atrophy, Tourette's syndrome, Friedrich's Ataxia, Machado-Joseph's disease, lewy body dementia, dystonia, Progressive supranuclear palsy, and frontotemporal dementia.

The degenerative brain disease of the present invention is a disease that causes various symptoms as degenerative changes appear in nerve cells of the central nervous system, wherein in most cases, the onset of diseases begins slowly, and symptoms appear after long periods of normal functioning after birth. In addition, once onset occurs, the disease continues to progress over several years or decades until death, and there is often a family history.

In the present invention, the degenerative brain disease is preferably Alzheimer's disease.

The purpose of diagnosing degenerative brain disease may be detection of target miRNAs and/or diagnosis of diseases through the detection. ‘Target miRNA’ refers to any kind of miRNA to be detected, and is annealed or hybridized with a primer or probe under hybridization, annealing, or amplification conditions.

According to an embodiment of the present invention, the target miRNA may be any one or more selected from the group consisting of mmu-miR-1187, mmu-miR-1306-3p, mmu-miR-7038-3p, mmu-miR-5113, mmu-miR-669n, mmu-miR-669c-5p, mmu-miR-365-2-5p, mmu-miR-3095-3p, mmu-miR-365-1-5p, mmu-miR-1931, mmu-miR-1306-5p, mmu-miR-7001-5p, mmu-miR-23a-5p, mmu-miR-574-5p, mmu-miR-3061-5p, mmu-miR-8117, mmu-miR-15a-3p, mmu-miR-665-5p, mmu-miR-669m-5p, mmu-miR-466m-5p, mmu-miR-668-5p, mmu-miR-6997-5p, and mmu-miR-7684-5p. In addition, human miRs corresponding to the above miRs may be included. For example, the human miRs may be any one or more selected from the group consisting of hsa-miR-1306-3p, hsa-miR-365b-5p, hsa-miR-365a-5p, hsa-miR-1306-5p, hsa-miR-23a-5p, hsa-miR-574-5p, hsa-miR-15a-3p, hsa-miR-665, and hsa-miR-668-5p.

The expression level of the miRNA may be increased in a patient group with neurodegenerative diseases.

More preferably, the miRNA is at least any one selected from the group consisting of mmu-miR-1187, hsa-miR-23a-5p, hsa-miR-365a-5p, and hsa-miR-574-5p.

The composition for detecting miRNA and/or the composition for diagnosing neurodegenerative diseases according to the present invention may detect and/or diagnose any miRNA from a biological sample. The term “biological sample” means any sample containing any RNA. The biological sample may be any tissue or body fluid obtained from a subject.

The biological sample comprises, but not limited to, a subject's sputum, blood, serum, plasma, blood cells (for example, white blood cells), tissues, biopsy samples, smear samples, rinse samples, swab samples, cell-containing body fluids, mobile nucleic acid, urine, peritoneal fluid and pleural fluid, hippocampus, cerebrospinal fluid, feces, lacrimal fluid or cells therefrom. Biological samples may also include tissue sections taken for histological purposes, i.e., frozen or fixed sections or microdissected cellular or extracellular portions thereof. The biological sample may be obtained by a method that does not harm a subject.

Preferably, the sample may be mixed in a buffer solution containing a surfactant to be provided.

In particular, the composition and/or kit according to the present invention is capable of detecting miRNAs at very low concentrations due to significantly excellent detection efficiency. Accordingly, the composition and/or kit may be used for detection by targeting miRNAs present in a trace amount in the human body.

The composition may further comprise a surfactant separately. In other words, the liposome comprising a first probe having a hairpin structure in which a reporter is conjugated to the 5′ end and a quencher is conjugated to the 3′ end and having a sequence complementary to a target miRNA; and the liposome comprising a second probe having a hairpin structure and having a sequence complementary to the first probe are present separately in the hydrogel, but treatment with a surfactant for detection of the target nucleic acid sequence degrades the membranes of the liposomes to perform the reaction. It may be provided in the form of a buffer solution containing a surfactant.

The present invention provides a kit containing a hydrogel for detecting miRNAs, wherein the hydrogel comprises a liposome comprising a first probe having a hairpin structure in which a reporter is conjugated to the 5′ end and a quencher is conjugated to the 3′ end and having a sequence complementary to a target miRNA; and a liposome comprising a second probe having a hairpin structure and having a sequence complementary to the first probe.

The present invention provides a kit containing a hydrogel for diagnosing neurodegenerative diseases, wherein the hydrogel comprises a liposome comprising a first probe having a hairpin structure in which a reporter is conjugated to the 5′ end and a quencher is conjugated to the 3′ end and having a sequence complementary to a target miRNA; and a liposome comprising a second probe having a hairpin structure and having a sequence complementary to the first probe.

In the kit, an optimal amount of reagents to be used in a particular reaction may be easily determined by those skilled in the art having the knowledge of the disclosure herein. Typically, the kit of the present invention is manufactured in a separate package or compartment including the above described components.

In addition, the kit may further comprise instructions for use and other tools or equipment necessary for detection. For example, the kit may further comprise a surfactant separately.

The present invention provides a method for detecting miRNAs comprising reacting a sample with a hydrogel, wherein the hydrogel comprises a liposome comprising a first probe having a hairpin structure in which a reporter is conjugated to the 5′ end and a quencher is conjugated to the 3′ end and having a sequence complementary to a target miRNA; and a liposome comprising a second probe having a hairpin structure and having a sequence complementary to the first probe.

In the method for detecting miRNAs according to the present invention, the sample may be contained in a buffer solution containing a surfactant. Accordingly, the buffer solution containing the surfactant may degrade the membrane of the liposome, and the catalytic hairpin assembly (CHA) reaction may be performed through a reaction with the first probe released from the liposome, thereby exhibiting a fluorescence change, or the like.

The detection method of the present invention may further comprise a step of visually confirming a change in fluorescence of a reactant; or measuring a change in fluorescence of a reactant. The measurement of the change in fluorescence may be a conventional fluorescence measurement method in which a measurement wavelength of a fluorescence device is fixed and measured. Specifically, the change in fluorescence may be confirmed by fixing the measurement wavelength of the fluorescence device. For example, FAM fluorescence may be measured using a method of measuring the fluorescence intensity of a reactant at a wavelength of ex; 495/em; 520 and observing the fluorescence change at intervals of 5 to 10 minutes for 1 to 2 hours.

The present invention provides a method for diagnosing neurodegenerative diseases comprising reacting a sample with a hydrogel, wherein the hydrogel comprises a liposome comprising a first probe having a hairpin structure in which a reporter is conjugated to the 5′ end and a quencher is conjugated to the 3′ end and having a sequence complementary to a target miRNA; and a liposome comprising a second probe having a hairpin structure and having a sequence complementary to the first probe.

The diagnostic method according to the present invention aims to detect miRNAs showing expression differences between a normal group and a group with neurodegenerative diseases and to diagnose through the detection.

Specifically, the target miRNA may be any one or more selected from the group consisting of mmu-miR-1187, mmu-miR-1306-3p, mmu-miR-7038-3p, mmu-miR-5113, mmu-miR-669n, mmu-miR-669c-5p, mmu-miR-365-2-5p, mmu-miR-3095-3p, mmu-miR-365-1-5p, mmu-miR-1931, mmu-miR-1306-5p, mmu-miR-7001-5p, mmu-miR-23a-5p, mmu-miR-574-5p, mmu-miR-3061-5p, mmu-miR-8117, mmu-miR-15a-3p, mmu-miR-665-5p, mmu-miR-669m-5p, mmu-miR-466m-5p, mmu-miR-668-5p, mmu-miR-6997-5p, and mmu-miR-7684-5p. In addition, human miRs corresponding to the above miRs may be included. For example, the human miR may be any one or more selected from the group consisting of hsa-miR-1306-3p, hsa-miR-365b-5p, hsa-miR-365a-5p, hsa-miR-1306-5p, hsa-miR-23a-5p, hsa-miR-574-5p, hsa-miR-15a-3p, hsa-miR-665, and hsa-miR-668-5p.

In other words, the first probe according to the present invention comprises a sequence complementary to the above-described miRNA.

The miRNA corresponds to a miRNA of which expression level is increased in the patient group suffering from neurodegenerative diseases compared to the normal group.

In other words, the present invention may comprise the method for diagnosing neurodegenerative diseases comprising reacting a sample with a hydrogel, wherein the hydrogel comprises a liposome comprising a first probe having a hairpin structure in which a reporter is conjugated to the 5′ end and a quencher is conjugated to the 3′ end and having a sequence complementary to a target miRNA; and a liposome comprising a second probe having a hairpin structure and having a sequence complementary to the first probe, the method comprising:
(a) reacting a sample with a hydrogel, the hydrogel comprises a liposome comprising a first probe having a hairpin structure in which a reporter is conjugated to the 5′ end and a quencher is conjugated to the 3′ end and having a sequence complementary to a target miRNA; and a liposome comprising a second probe having a hairpin structure and having a sequence complementary to the first probe;
(b) visually confirming a change in fluorescence of a reactant with the sample or measuring the change in fluorescence; and
(c) diagnosing the onset of the neurodegenerative diseases by confirming the change in fluorescence or measuring the change in fluorescence.

The present invention provides a microfluidic chip for detecting miRNAs comprising: an inlet;
a microtubule passage connecting the inlet to an outlet; and
sensing parts of the outlet configured to be branched from the microtubule passage, wherein the sensing part comprises a hydrogel, wherein the hydrogel comprises a liposome comprising a first probe having a hairpin structure in which a reporter is conjugated to the 5′ end and a quencher is conjugated to the 3′ end and having a sequence complementary to a target miRNA; and a liposome comprising a second probe having a hairpin structure and having a sequence complementary to the first probe.

The present invention provides a microfluidic chip for detecting neurodegenerative diseases comprising: an inlet;
a microtubule passage connecting the inlet to an outlet; and
sensing parts of the outlet configured to be branched from the microtubule passage, wherein the sensing part comprises a hydrogel, wherein the hydrogel comprises a liposome comprising a first probe having a hairpin structure in which a reporter is conjugated to the 5′ end and a quencher is conjugated to the 3′ end and having a sequence complementary to a target miRNA; and a liposome comprising a second probe having a hairpin structure and having a sequence complementary to the first probe.

The microfluidic chip has the ability to simultaneously perform various experimental conditions by flowing fluid through the microfluidic channel. Specifically, a microchannel may be manufactured using a substrate (or chip material) such as plastic, glass, silicon, or the like, so that a fluid (for example, a liquid sample) may be moved through the channel, and then reaction and detection may be performed through a plurality of sensing parts in the microfluidic chip.

The above-described structure is explained as follows based on FIG. 15.

A microfluidic chip (001) is provided with an inlet (002) located therein, and includes a microtubule passage (003) connecting the sample and/or the surfactant to move from the inlet to the outlet. The above microtubule passage allows fluids (for example, samples (including target nucleic acids, and the like) and surfactants, and the like) to pass to the outlet. The microtubule passage (003) has a branch (004).

This branch is formed with an angle of approximately 45° with each extension line in the direction of fluid flow from the inlet, and is connected to each of two sensing parts 005. In other words, the microfluidic chip includes two sensing parts 005 of the branched outlet.

The sensing part comprises the hydrogel disposed therein according to the present invention. The hydrogel exhibits a change in fluorescence as the liposomes are degraded due to the sample and/or the surfactant to be administered, thereby performing the CHA reaction.

The sensing part may comprise a first sensing part for detecting a house-keeping gene and a second sensing part for detecting a target gene. The house-keeping gene of the first sensing part refers to a gene capable of being easily and generally used to normalize gene expression patterns such as glyceraldehyde-3-phosphate dehydrogenase (GAPDH), Cypl, albumin, actin, tubulin, cyclophiiin hypoxantine phosphoribosyltransferase (HRPT), L32,28S, U6, 18S, and the like. The house-keeping gene may be used to correct the signal of the target gene, thereby correcting a difference in an amount of quantitative genes among individuals.

In other words, the first probe and the second probe of the first sensing part may contain sequences for detecting the house-keeping gene.

The second sensing part may be used to detect a target miRNA. The first probe and the second probe of the second sensing part may contain sequences for detecting a target miRNA.

According to an embodiment of the present invention, a sensing part having a size of approximately 8 mm was formed through swelling of a 6 mm hydrogel, and a height thereof was set to approximately 1 mm.

The entire chip had a structure set to approximately 65 mm in width and 25 mm in length.

Accordingly, the microfluidic chip preferably has a size of about 50 to 100 mm in a (horizontal) direction in which the fluid flows, and preferably has a size of about 15 to 40 mm in a vertical direction. The hydrogel preferably has a diameter size of approximately 4 mm to 12 mm, and a height of approximately 0.5 mm to 2 mm.

The present invention also provides a composition for diagnosing neurodegenerative diseases comprising: an agent capable of measuring the expression level of any one or more miRNAs selected from the group consisting of mmu-miR-1187, has-mirR-365a-5p, has-mirR-23a-5p, and has-mirR-574-5p.

“Diagnosis” includes determining the susceptibility of a subject to a particular disease or condition, determining whether a subject currently has a particular disease or condition, determining the prognosis of a subject suffering from a particular disease or disorder; or therametrics (for example, monitoring the condition of the subject to provide information about treatment efficacy).

The step of measuring the miRNA expression level may be performed using any method known to those skilled in the art. As specific examples, the measurement methods may be PCR, ligase chain reaction (LCR), transcription amplification, autonomous sequence replication, and nucleic acid-based sequence amplification (NASBA) methods, and the like, but are not limited thereto. Here, the base sequences of the four miRNAs according to the present invention are known in databases such as NCBI, and the like, and thus those skilled in the art may use appropriate means required for measuring the miRNA expression level.

Each of the four markers is characterized by an increased expression level in a patient group with neurodegenerative diseases.

As used herein, “an agent for measuring the expression level of miRNA” refers to an agent capable of being specifically bound to recognize the miRNA or amplify the miRNA. As a specific example, the agent may be a primer or probe that specifically binds to the miRNA, but is not limited thereto, and those skilled in the art will be able to select an appropriate agent for the purpose of the invention.

The agent may be directly or indirectly labeled to measure the expression level of the gene. Specifically, ligands, beads, radionuclides, enzymes, substrates, cofactors, inhibitors, fluorescers, chemiluminescent materials, magnetic particles, haptens, dyes, and the like, may be used as the label, but the label is not limited thereto. Specifically, examples of the ligand comprise biotin, avidin, streptavidin, and the like, examples of the enzyme comprise luciferase, peroxidase, beta-galactosidase, and the like, and examples of the fluorescer comprise fluorescein, coumarin, rhodamine, phycoerythrin, and sulforhodamic acid chloride (Texas red), and the like, but these materials are not limited thereto. Most known labels may be used as the detectable label, and those skilled in the art will be able to select appropriate labels depending on the purpose of the invention.

Term ‘primer’ is a base sequence with a short free 3′ hydroxyl group, which refers to a short sequence capable of forming a base pair with a complementary template and functioning as a starting point for copy of template strand. In the present invention, the primers used for miRNA amplification may be a single-stranded oligonucleotide capable of acting as a starting point for template-directed DNA synthesis under suitable conditions (for example, four different nucleoside triphosphates and polymerases such as DNA and RNA polymerases or reverse transcriptase) in an appropriate buffer and at an appropriate temperature, wherein an appropriate length of the primer may vary depending on the purpose of use. The primer sequence does not have to be completely complementary to the polynucleotide of the miRNA of the gene or complementary polynucleotide thereto, and may be used as long as it is sufficiently complementary to hybridize.

Term ‘probe’ refers to a labeled nucleic acid fragment or peptide capable of specific binding to miRNA. As specific examples, the probe may be prepared into oligonucleotide probes, single stranded DNA probes, double stranded DNA probes, RNA probes, oligonucleotide peptide probes, polypeptide probes, and the like.

The present invention also provides a kit for diagnosing neurodegenerative diseases comprising: an agent capable of measuring the expression level of any one or more miRNAs selected from the group consisting of mmu-miR-1187, has-mirR-365a-5p, has-mirR-23a-5p, and has-mirR-574-5p.

As a specific example, the kit may be an RT-PCR kit, but is not limited thereto as long as it is able to measure the expression level of miRNAs.

Here, the RT-PCR kit may be a kit including essential elements required to perform RT-PCR. For example, the RT-PCR kit may comprise, in addition to each primer specific for the gene, a test tube or other suitable containers, a reaction buffer (various pH levels and magnesium concentrations), deoxynucleotides (dNTPs), dideoxynucleotides (ddNTPs), enzymes such as Taq-polymerase and reverse transcriptase, DNase and RNAse inhibitors, DEPC-water, sterile water, and the like. In addition, the kit may also comprise a primer pair specific to a gene used as a quantitative control.

Further, in order to provide information necessary for diagnosing neurodegenerative diseases, the present invention provides a method for diagnosing neurodegenerative diseases, comprising: providing a sample derived from a subject in need of a diagnosis of a degenerative brain disease;

measuring the expression level of any one or more miRNAs selected from the group consisting of mmu-miR-1187, has-mirR-365a-5p, has-mirR-23a-5p, and has-mirR-574-5p in the sample; and

comparing a concentration of a detected marker with a test result of a normal control group to diagnose the degenerative brain disease of the subject under examination.

### Advantageous Effects

When the detection system according to the present invention is used, effective real-time diagnosis efficiency may be exhibited while problems such as noise are minimized. In particular, excellent diagnostic effects on neurodegenerative diseases including Alzheimer's disease are exhibited by detecting and diagnosing miRNA present in a trace amount with high detection efficiency.

### DESCRIPTION OF DRAWINGS

FIG. 1 shows microarray analysis results of 25 up-regulated miRNAs and 70 down-regulated miRNAs as miRNAs showing expression differences in degenerative brain disease models.

FIG. 2 shows changes in expression of mmu-miR-1187, mmu-miR-23a-5p, mmu-miR-365-1-5p, and mmu-miR-574-5p.

FIG. 3 is a schematic diagram of the catalytic hairpin assembly (CHA) reaction according to the present invention.

FIG. 4 shows the reaction results of probe designs.

FIG. 5 shows fluorescent results of reactions between the target sequence and the probe used for the CHA reaction.

FIG. 6 shows the results of confirming that the probes are encapsulated in the liposomes.

FIG. 7 shows a process for producing a hydrogel according to the present invention and production results thereof.

FIG. 8 is a schematic diagram of a microfluidic chip according to the present invention.

FIG. 9 shows the configuration for the entire reaction of the microfluidic chip according to the present invention and the specific configuration of a sensing part therein.

FIG. 10 shows that the probes encapsulated in the liposomes according to the present invention are released by treatment with a surfactant and participate in a reaction.

FIG. 11 shows the diffusion change of the probe in the optimized hydrogel.

FIG. 12 shows the sensitivity evaluation results of a hydrogel system including probe-supported liposomes.

FIG. 13 shows detection of target miRNAs of the present invention confirmed from RNAs isolated from hippocampus tissues of 5XFAD mice.

FIG. 14 shows detection of the target miRNAs of the present invention confirmed from RNAs isolated from the blood of 5XFAD mice.

FIG. 15 is a schematic diagram of components of the microfluidic chip according to the present invention.

### BEST MODE

Hereinafter, the present disclosure will be described in more detail through Examples. However, these Examples are provided to illustrate the present disclosure by way of example, and the scope of the present disclosure is not limited to these Examples.

### <Example 1> Discovery of Biomarkers

### (1) Experimental Animal Model

As experimental animal models, 4-month-old 5XFAD Alzheimer's dementia mouse models (n=5) were used, and as a control group, 4-month-old wild-type mice were used. 5XFAD Alzheimer's dementia mice were female heterozygous 5XFAD transgenic mice (B6SJL/mice background; 4 months old). 5XFAD mice were confirmed by genotyping, and wild-type (WT) littermates of 5XFAD mice were used as a control group.

### (2) Plasma Collection

Mice were anesthetized using Avertin (2,2,2-tribromoethanol, Sigma aldrich), and then blood was collected from the orbital venous plexus of the anesthetized mice using a disposable micro-hematocrit capillary tube (Marienfeld Superior). The collected blood was centrifuged for 15 minutes at 13,000 rpm at 4° C. after adding 2 μL of heparin (0.1 mg/mL) to prevent coagulation.

### (3) Mouse Brain Tissue Extraction

The brain was extracted from the mouse after blood collection, and then the extracted brain of the mouse was carefully separated into the hippocampus, subiculum, and frontal cortex parts using brain matrices and dorco razor blades. Each part was determined by referring to Paxinos and Franklin's the Mouse Brain in Stereotaxic Coordinates, and the hippocampus was a part excised from 1 mm away from bregma in a caudal direction (the junction of the sagittal and coronal sutures) to 3 mm (−1.00 mm to −3.00 mm from bregma). The subiculum corresponded to a part excised from 2 mm away from bregma in a caudal direction to 4 mm (−2.00 mm to −4.00 mm from bregma). The frontal cortex was a part excised from 2 mm away from bregma in a head direction to 4 mm (+2.00 mm to +4.00 mm from bregma).

(4) miRNA Extraction

The extracted mouse brain tissue was pulverized using a grinder equipped with a sterile pestle, and miRNAs were extracted from the tissue using the RNeasy Mini kit (Qiagen). Plasma RNA was extracted using the ExoRNeasy Maxi kit (Qiagen). The concentration of extracted RNA was measured using a spectrophotometer (Nanodrop 2000, Thermo).

### (5) Microarray Analysis

RNA samples extracted from tissues were subjected to microarray analysis at a concentration of 100 ng/pL and analyzed by Macrogen (Korea). Then, miRNA candidate groups targeting disease detection were selected by analyzing the difference in miRNA expression levels between the experimental group and the control group.

(6) Quantitative Reverse Transcriptase PCR (qRT-PCR)

RNAs extracted from tissue and plasma were subjected to reverse transcription using the miScript II RT kit to synthesize cDNA, wherein PCR was performed according to the protocol of the miScript SYBR Green PCR Kit (Qiagen). The mRNA analysis was performed on CFX96™ Real-Time equipment (Bio-rad), and all experiments were repeated 3 times. Each sample was subjected to normalization with U6 (a housekeeping gene) to obtain quantitative results.

Sequence information used, miR information, and fold change levels are shown in Table 1 below.

TABLE 1
Sequence Information

| | Fold | | | |
|---|---|---|---|---|
| | Change | | | |
| | Log2 (5X | | Hsa-miR | |
| Transcript ID | FAD/WT) | miR-Sequence | Transcript ID | sequence |
| mmu-miR-1187 | 6.66 | UAUGUGUGUGUGUAUGUGUGUAA | | |
| | | (SEQ ID NO: 1) | | |
| mmu-miR-1306- | 3.65 | ACGUUGGCUCUGGUGGUGAUG | hsa-miR- | ACGUUGGCUCUGGUGGUG |
| 3p | | (SEQ ID NO: 2) | 1306-3p | (SEQ ID NO: 24) |
| mmu-miR-7038- | 3.58 | CACUGCUCCUGCCUUCUUACAG | | |
| 3p | | (SEQ ID NO: 3) | | |
| mmu-miR-5113 | 3.23 | ACAGAGGAGGAGAGAGAUCCUGU | | |
| | | (SEQ ID NO: 4) | | |
| mmu-miR-669n | 3 | AUUUGUGUGUGGAUGUGUGU | | |
| | | (SEQ ID NO: 5) | | |
| mmu-miR-669c- | 3 | AUAGUUGUGUGUGGAUGUGUGU | | |
| 5p | | (SEQ ID NO: 6) | | |
| mmu-miR-365- | 2.83 | AGGGACUUUCAGGGGCAGCUGUG | hsa-miR- | AGGGACUUUCAGGGGCAGCUGU |
| 2-5p | | (SEQ ID NO: 7) | 365b-5p | (SEQ ID NO: 25) |
| mmu-miR-3095- | 2.79 | AAGCUUUCUCAUCUGUGACACU | | |
| 3p | | (SEQ ID NO: 8) | | |
| mmu-miR-365- | 2.73 | AGGGACUUUUGGGGGCAGAUGUG | hsa-miR- | AGGGACUUUUGGGGGCAGAUGUG |
| 1-5p | | (SEQ ID NO: 9) | 365a-5p | (SEQ ID NO: 26) |
| mmu-miR-1931 | 2.67 | AUGCAAGGGCUGGUGCGAUGGC | | |
| | | (SEQ ID NO: 10) | | |
| mmu-miR-1306- | 2.61 | CACCACCUCCCCUGCAAACGUCC | hsa-miR- | CCACCUCCCCUGCAAACGUCCA |
| 5p | | (SEQ ID NO: 11) | 1306-5p | (SEQ ID NO: 27) |
| mmu-miR-7001- | 2.46 | AGGCAGGGUGUGAGCGUGAGCAU | | |
| 5p | | (SEQ ID NO: 12) | | |
| mmu-miR-23a- | 2.38 | GGGGUUCCUGGGGAUGGGAUUU | hsa-miR- | GGGGUUCCUGGGGAUGGGAUUU |
| 5p | | (SEQ ID NO: 13) | 23a-5p | (SEQ ID NO: 28) |
| mmu-miR-574- | 2.26 | UGAGUGUGUGUGUGUGAGUGUGU | hsa-miR- | UGAGUGUGUGUGUGUGAGUGUGU |
| 5p | | (SEQ ID NO: 14) | 574-5p | (SEQ ID NO: 29) |
| mmu-miR-3061- | 2.2 | CAGUGGGCCGUGAAAGGUAGCC | | |
| 5p | | (SEQ ID NO: 15) | | |
| mmu-miR-8117 | 2.17 | GCUCGUGUGGAACAGAAGGGG | | |
| | | (SEQ ID NO: 16) | | |
| mmu-miR-15a- | 2.17 | CAGGCCAUACUGUGCUGCCUCA | hsa-miR- | CAGGCCAUAUUGUGCUGCCUCA |
| 3p | | (SEQ ID NO: 17) | 15a-3p | (SEQ ID NO: 30) |
| mmu-miR-665- | 2.17 | AGGGGCCUCUGCCUCUAUCCAGG | hsa-miR-665 | ACCAGGAGGCUGAGGCCCCU |
| 5p | | AUU | | (SEQ ID NO: 31) |
| | | (SEQ ID NO: 18) | | |
| mmu-miR-669m- | 2.13 | UGUGUGCAUGUGCAUGUGUGUAU | | |
| 5p | | (SEQ ID NO: 19) | | |
| mmu-miR-466m- | 2.13 | UGUGUGCAUGUGCAUGUGUGUAU | | |
| 5p | | (SEQ ID NO: 20) | | |
| mmu-miR-668- | 2.12 | GUAAGUGUGCCUCGGGUGAGCAUG | hsa-miR- | UGCGCCUCGGGUGAGCAUG |
| 5p | | (SEQ ID NO: 21) | 668-5p | (SEQ ID NO: 32) |
| mmu-miR-6997- | 2.03 | UAACAGGCUGGAGAGGUGCAGA | | |
| 5p | | (SEQ ID NO: 22) | | |
| mmu-miR-7684- | 2.01 | UCUGGGAAGCCUGGGCAGCAG | | |
| 5p | | (SEQ ID NO: 23) | | |

### (7) Experimental Results

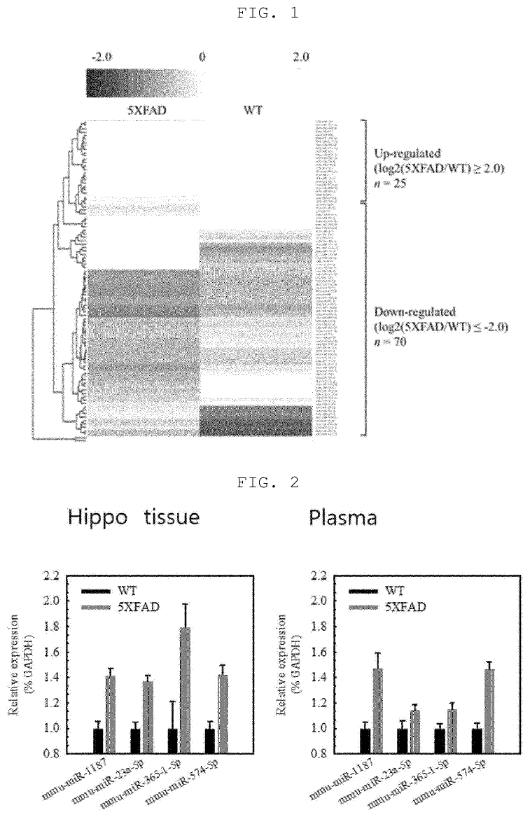

The experimental results through the microarray analysis are shown in FIG. 1. As could be confirmed in FIG. 1, 25 kinds of up-regulated miRNAs and 70 kinds of down-regulated miRNAs were identified as miRNAs.

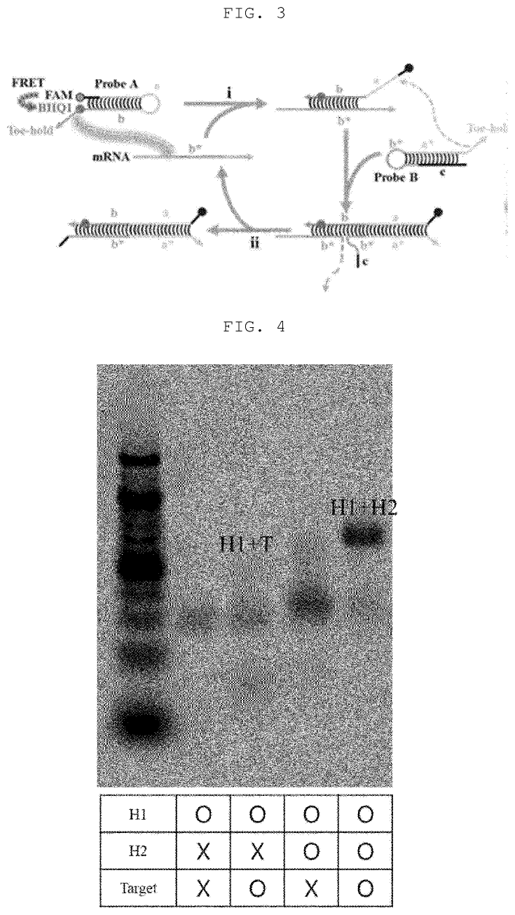

Among the up- and down-regulated miRNAs, the up-regulated miRNAs were subjected to qRT-PCR, and results thereof are shown in FIG. 2 and Table 1.

Table 1 shows fold changes and matching human miRNAs of upregulated factors.

In particular, as shown in FIG. 2 upon reviewing the microarray results, mmu-miR-1187 showing the highest expression level among miRNA candidates of which expression level increased more than 1.5 times, and three kinds with the same human miRNA sequence, i.e., mmu-miR-23a-5p, mmu-miR-365-1-5p, and mmu-miR-574-5p, were selected as miR candidates.

Human miRNAs respectively corresponding to mmu-miR-23a-5p, mmu-miR-365-1-5p and mmu-miR-574-5p are hsa-miR-23a-5p, hsa-miR-365a-5p and hsa-miR-574-5p.

### <Example 2> Design of Self-Signal Amplifying DNA Probe For mRNA Detection

The reaction principle of catalytic hairpin assembly (CHA) according to the present invention is shown in FIG. 3. FIG. 3 schematically shows the CHA circuit composed of probe A (PA) and probe B (PB) for signal amplification in a hydrogel. The PA strand of the circuit is modified at each end with a fluorophore (FAM) and a quencher (BHQ1), respectively, and the target mRNA initiates fluorescence recovery (i) and catalyzes the assembly of PA and PB via a toehold-mediated hairpin DNA circuit (ii).

Probes A and B each having a hairpin structure were designed. The base sequences of the designed probes are shown in Table 2.

TABLE 2

| | Length | |
|---|---|---|
| name | (nt) | Sequences (5′ to 3′) |
| H1 | 54 | [FAM] tgtgtgtgtgagtgtgggatcgaaagt |
| | | gtgtgcacactcacacacacacactca |
| | | [BHQ1] |
| | | (SEQ ID NO: 33) |
| H2 | 58 | Tgtgggatcgaaagtgtgtgtgtgtgagtgtgc |
| | | acacactttcgatcccacactcaca |
| | | (SEQ ID NO: 34) |
| Target-574 | 23 | Tgagtgtgtgtgtgtgagtgtgt |
| | | (SEQ ID NO: 35) |
| 1MS-574 | 23 | Tgagtgtgggtgtgtgagtgtgt |
| | | (SEQ ID NO: 36) |
| 2MS-574 | 23 | Tgagtctgggtgtgtgagtgtgt |
| | | (SEQ ID NO: 37) |

*Target: Target sequence,
**1MS: 1 base mismatched sequence,
***2MS: 2 base mismatched sequence

The probes were designed according to the theory of non-enzymatic fluorescence signal amplification. 6-Carboxylfluorescein (6-FAM) was bound to the 5′ end of the nucleotide sequence of probe A. A quencher blackhole quencher-1 (BHQ1) was bound to the 3′ end of the nucleotide sequence of probe A. The probes were boiled at 90° C. for 5 minutes and then slowly cooled at room temperature to annealing. All probes were stored frozen until use.

Mutual binding of the designed probe groups was confirmed by performing polyacrylamide gel electrophoresis (PAGE). The electrophoresis gel was prepared with 10% acrylamide and run for 90 minutes under a voltage of 80 V using 1× TBE buffer. Then, the DNA was stained with GelRed® for 10 minutes to mark the location of the DNA, and then photographed with Gel-Doc (Bio-Rad Laboratories, Inc.) system.

The above reaction was confirmed by gel electrophoretic analysis using the synthesized probe set. Specifically, mutual binding of the designed probe groups was confirmed by performing polyacrylamide gel electrophoresis (PAGE). The electrophoresis gel was prepared with 10% acrylamide and run for 90 minutes under a voltage of 80 V using 1×TBE buffer. Then, the DNA was stained with GelRed® for 10 minutes to mark the location of the DNA, and then photographed with Gel-Doc (Bio-Rad Laboratories, Inc.) system.

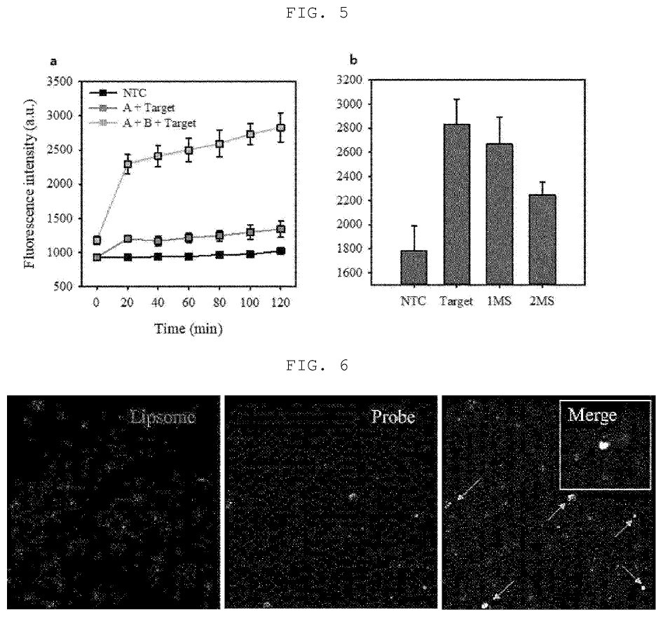

Results thereof are shown in FIG. 4.

As shown in FIG. 4, it could be confirmed that the reaction proceeded sequentially by the above probe set at room temperature.

The forms and results of these reactions were confirmed in more detail through fluorescence analysis, and results thereof are shown in FIG. 5.

According to FIG. 5a, it was shown that due to the role of probe B, a larger amount of fluorescence signal was generated within the same time (A+B+Target compared to A+Target), and that the fluorescence signal was stably maintained in the target-free condition (A+B).

In addition, as shown in FIG. 5b, as a result of confirming the selectivity of the detection probe using the Experimental Group DNA (Control) in which one (1 MS) or 2 (2 MS) nucleotide sequence was replaced from the target miRNA, the reaction with the target miRNA showed the highest fluorescence, which had a large difference from the fluorescence of the control gene reaction.

It was confirmed from these results that the catalytic hairpin assembly (CHA) system according to the present invention exhibits excellent effects in detecting target genes.

### <Example 3> Preparation of Polyethylene Glycol Diacrylate (PEGDA)

Polyethylene glycol (PEG, 60 g) was dissolved in 75 mL of dichloromethane (DCM). It was confirmed that the solution became transparent, and then 7 mL of N,N-diisopropylethylamine (DIPEA) was added to the solution. While maintaining the glass containing the solution at 4° C., 6.5 mL of acryloyl chloride was added. This reaction was performed in a shaded place for 8 to 12 hours while refluxing under nitrogen. Diethyl ether (1 L) was added to the reaction mixture to obtain a precipitate, which was dried in a vacuum chamber. The dried compound was additionally dissolved in 75 mL of dichloromethane and 500 mL of 2 mol potassium carbonate (K₂CO₃) and reacted for 8 to 12 hours, followed by precipitation by adding 1 L of diethyl ether to thereby obtain polyethylene glycol diacrylate. Then, the precipitate was dried in a vacuum chamber to obtain a resulting product in a powder form.

This product was prepared to be used as a hydrogel of PEGDA material.

### <Example 4> Preparation of Probe-Supported Liposome

Liposomes were prepared by the conventional lipid film hydration method. To 10 mL of chloroform solution, 7 mg of phosphatidylcholine (PC), 0.7 mg of dioleoyl-3-trimethylammonium propane (DOTAP), and 1.95 mg of cholesterol (5-cholesten-3β-ol) were dissolved. A thin lipid film was prepared by evaporating the solvent using a rotary vacuum evaporator at room temperature. After adding 1 mL of 10 nM oligonucleotide solution in TE buffer to the lipid film, the lipid film was separated from the glass using a vortex mixer. The solution was stored at 4° C. for 8 to 12 hours. In order to remove unencapsulated oligonucleotides, the solution was filtered through an Amicon centrifugal filter at 4° C. and 4000 rpm for 60 minutes.

In order to confirm that the prepared probes were encapsulated in the liposomes, DNA probes with green fluorescence (FAM) were encapsulated in the liposomes and observed through a fluorescence microscope using a liposome dye (dye_red).

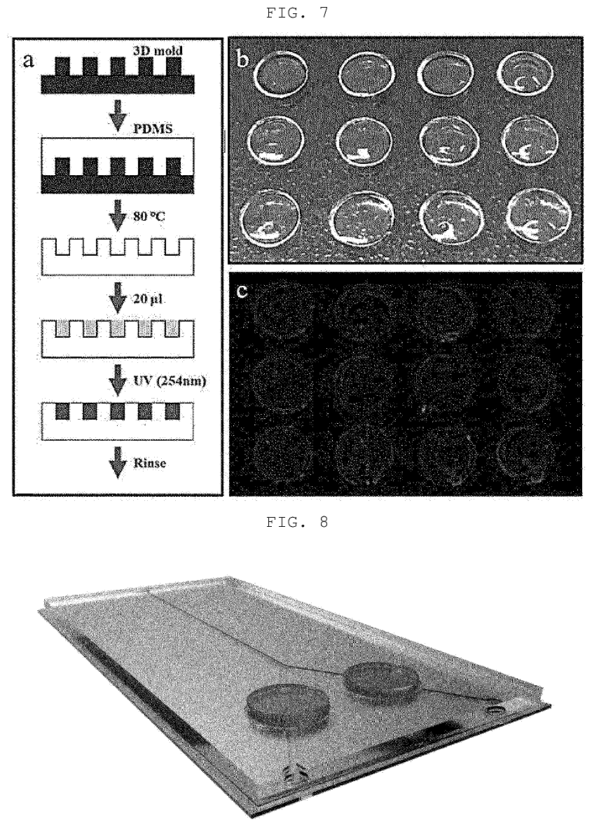

Results thereof are shown in FIG. 6.

As could be confirmed in FIG. 6, two types of fluorescence were seen at the same location, thereby confirming that the probes were encapsulated in the liposomes.

### <Example 5> Production of Hydrogel Containing Probe-Supported Liposome

Polyethylene glycol diacrylate at a weight ratio of 20%, polyethylene glycol at a weight ratio of 20%, liposomes encapsulated with 10 picomoles of the probes, and 2-hydroxy-2-methylpropiophenone (HMPP) at a weight ratio of 0.1% were combined. The prepared solution was exposed to an ultraviolet lamp (365 nm) for about 2 minutes to produce a hydrogel through photopolymerization. The produced hydrogel was soaked in sterile water (DW) for 2 hours to remove non-encapsulated liposomes.

The above overall process and confirmation results are shown in FIG. 7.

FIG. 7a shows the entire process of producing the PEGDA hydrogel, and FIGS. 7b and 7c show images of the produced hydrogel.

### <Example 6> Manufacturing of Microfluidic Chip

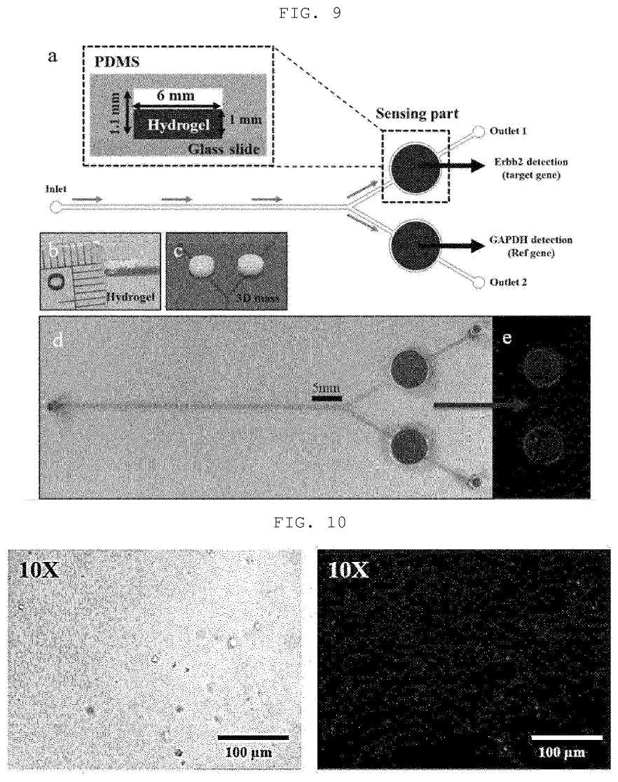

A casting mold (height: 100 μm) was manufactured by fabricating patterns using SU-8 photosensitive resin on a silicon wafer, as shown in FIG. 8. A structure having a diameter of 6 millimeters and a height of 1 millimeter produced by a 3D printer was attached to the manufactured casting mold, then liquid polydimethylsiloxane (PDMS) was solidified in the casting mold to form a chip, and the chip was attached to a slide glass, thereby manufacturing a microfluidic channel device.

The produced hydrogel was placed on the device and used for miRNA analysis.

Specifically, the analysis principle using the microfluidic chip is shown in FIG. 9.

A sample and a surfactant introduced through an inlet are directed to sensing parts of an outlet through a microtubule passage. Thus, the liposomes are degraded to release the probes, and the reaction between the probes and the detection miRNAs results in a change in fluorescence.

FIG. 9a shows the size of an exemplary sensing part, and FIG. 9b to 9d show sizes and configurations thereof.

Specifically, the sensing part of approximately 8 mm was constructed through swelling of a 6 mm hydrogel, wherein a height thereof was set to about 1 mm.

The entire chip had a structure set to approximately 65 mm in width and 25 mm in length.

### <Example 7> Evaluation of Hydrogel Containing Probe-Supported Liposome

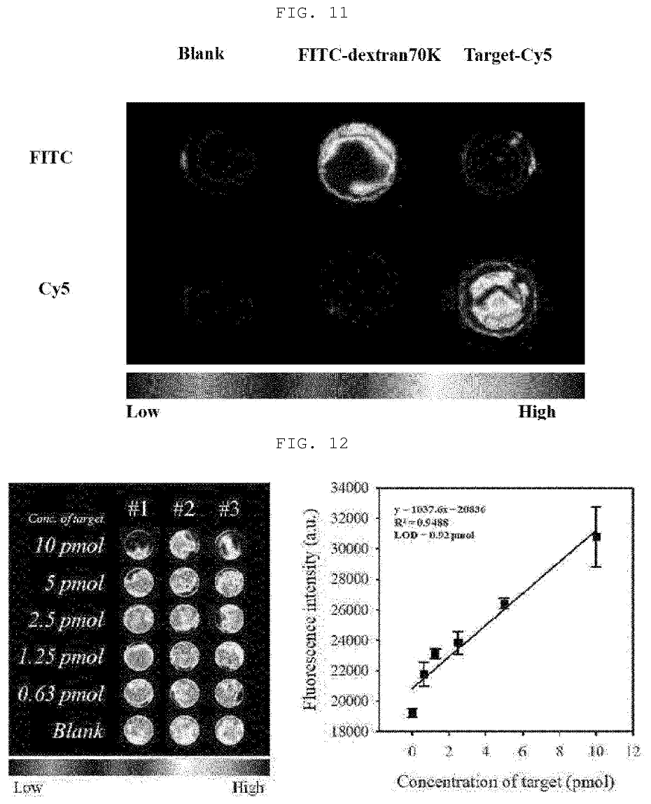

The hydrogel containing the probe-supported liposomes according to the present invention was confirmed through a microscope. Results thereof are shown in FIG. 10. As could be seen in FIG. 10, it was confirmed that the liposomes were encapsulated into the hydrogel.

### <Example 8> Optimization Progress of Hydrogel

Polyethylene glycol (PEG) may be added together at the time of producing hydrogel and may play a role in creating fine pores inside. Therefore, since the number of fine pores increases as the concentration increases, it is necessary to find the most suitable conditions for detection by adjusting the amount of fine pores (pores) inside the hydrogel.

Accordingly, the fluorescence change was confirmed under the condition of 20% PEG prepared in the present invention, and results thereof are shown in FIG. 11.

As could be seen in FIG. 11, it was confirmed that when mRNA diffusion was confirmed using 2 mg/mL FITC-Dextran70K (almost 12 nm of diameter) and 1 μM Cy5 modified oligonucleotides (20 nt), the most appropriate diffusion result was obtained.

### <Example 9> Evaluation of Sensitivity of Hydrogel System Containing Probe-Supported Liposomes

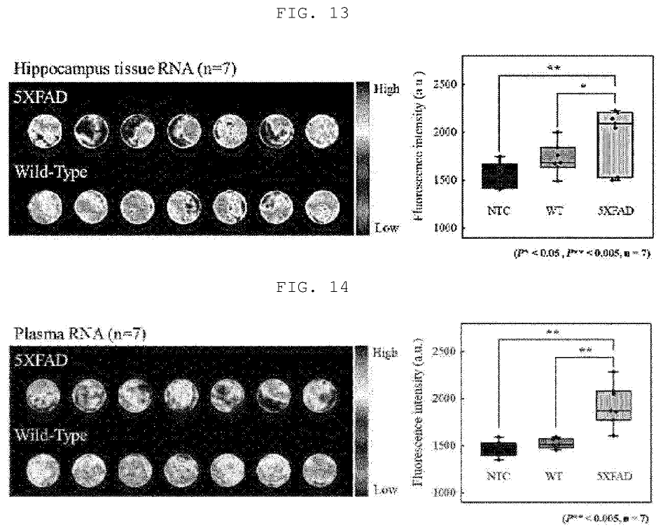

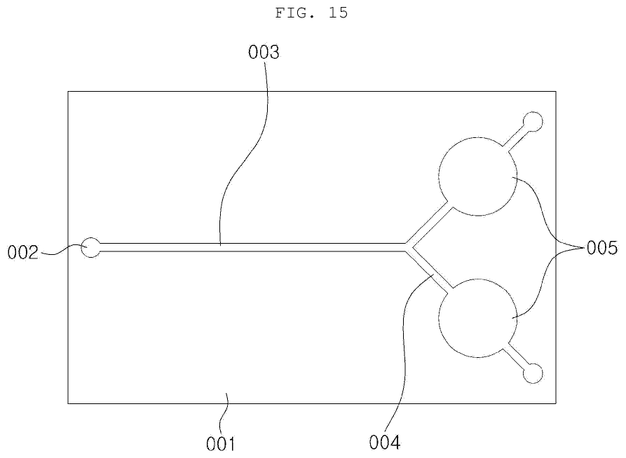

The hydrogel sensitivity of the probe was evaluated to determine the limit of detection. Specifically, detection sensitivity was measured while varying the target concentration from 0.63 pmol to 10 pmol, and results thereof are shown in FIG. 12.

As could be seen in FIG. 12, the detection limit was confirmed to be 0.92 pmol.

### <Example 10> miRNA Detection Result Extracted from Mouse Hippocampus Tissue

Changes in fluorescence response were confirmed by applying RNAs (250 ng/gel), which were extracted from the provided mouse hippocampus tissues (n=7), to the hydrogel for detection in Example 5.

Results thereof are shown in FIG. 13.

As could be confirmed in FIG. 13, the detection of the target miRNAs of the present invention was confirmed from the RNAs isolated from 5XFAD mice, and showed a significant fluorescence difference compared to the wild type.

From the above results, the detectability for miRNAs was confirmed.

### <Example 11> miRNA Detection Result Extracted from Mouse Blood

Changes in fluorescence response were confirmed by applying RNAs (100 ng/gel), which were extracted from the provided mouse blood (n=7), to the hydrogel for detection in Example 5.

Results thereof are shown in FIG. 14.

As could be confirmed in FIG. 14, the detection of the target miRNAs of the present invention was confirmed from the RNAs isolated from 5XFAD mice, and showed a significant fluorescence difference compared to the wild type.

From the above results, the detectability for miRNAs was confirmed.

### <Example 12> Application to Microfluidic System

Based on the microfluidic chip of Example 6, changes in miRNA expression in blood were confirmed using RNAs extracted from the hippocampus tissues or blood of the mice.

Specifically, the inside of the manufactured chip was made into a vacuum state by blocking the inlet of the chip and evacuating the gas inside the chip for 30 minutes using a vacuum chamber. After injecting about 100 μL of the sample solution through the inlet and reacting for 2 hours, fluorescence was measured with Gel-Doc system.

From the above description, those skilled in the art to which the present disclosure pertains will understand that the present disclosure may be embodied in other specific forms without changing the technical spirit or essential characteristics thereof. In this regard, it should be understood that the embodiments described above are illustrative in all respects and not restrictive. As the scope of the present disclosure, it should be construed that all changes or modifications derived from the meaning and scope of the claims to be described below and equivalents thereof rather than the above detailed description are included in the scope of the present disclosure.

### DETAILED DESCRIPTION OF MAIN ELEMENTS

001 MICROFLUIDIC CHIP
002 INLET
003 MICROTUBULE PASSAGE
004 BRANCH
005 SENSING PART

1.-19. (canceled)

20. A method for detecting microRNAs (miRNAs) comprising reacting a sample with a hydrogel,
wherein the hydrogel comprises a liposome comprising a first probe having a hairpin structure in which a reporter is conjugated to the 5′ end and a quencher is conjugated to the 3′ end and having a sequence complementary to a target miRNA; and a liposome comprising a second probe having a hairpin structure and having a sequence complementary to the first probe.

21. The method of claim 20, wherein particles of the hydrogel comprise at least one material selected from the group consisting of natural polymer, acrylic monomer or polymer, polyacrylamide-based monomer or polymer, phosphatidyl choline, hyaluronic acid-based monomer or polymer, carboxymethyl cellulose, alginate, chitosan, poly(e-caprolactone), poly(lactic acid), poly(glycolic acid), polyethylene glycol, hydroxyapatite, tricalcium phosphate, and mixtures thereof.

22. The method of claim 20, wherein the lipid constituting the liposome is any one or more selected from the group consisting of phospholipids, glycolipids, sterols, and cationic lipids.

23. The method of claim 20, wherein the reporter conjugated to the 5′ end is any one selected from the group consisting of ALEX-350, FAM, VIC, TET, CAL Fluor® Gold 540, JOE, HEX, CAL Fluor Orange 560, TAMRA, CAL Fluor Red 590, ROX, CAL Fluor Red 610, TEXAS RED, CAL Fluor Red 635, Quasar 670, CY3, CY5, CY5.5, and Quasar 705, and
the quencher conjugated to the 3′ end is any one selected from the group consisting of DABCYL, BHQ, ECLIPSE, and TAMRA.

24. The method of claim 20, wherein the method further comprises treating the liposome with a surfactant to degrade the membrane of the liposome.

25. The method of claim 20, wherein the sample is mixed in a buffer solution containing a surfactant.

26. The method of claim 25, wherein the surfactant in the sample mixture degrades the membranes of the liposome to initiate the reaction of the sample with the hydrogel.

27. The method of claim 25, wherein the surfactant is one selected from the group consist of cetyl trimethylammonium bromide, hexadecyl trimethyl ammonium bromide, dodecyl betaine, dodecyl dimethylamine oxide, 3-(N,N-dimethylpalmitylammonio)propane sulfonate), Tween 20, Tween 80, Triton-X-100, polyethylene glycol monooleyl ether, triethylene glycol monododecyl ether, octyl glucoside, and N-nonanoyl-N-methylglucamine.

28. A method for diagnosing a neurodegenerative disease comprising reacting a sample with a hydrogel,
wherein the hydrogel comprises a liposome comprising a first probe having a hairpin structure in which a reporter is conjugated to the 5′ end and a quencher is conjugated to the 3′ end and having a sequence complementary to a target miRNA; and a liposome comprising a second probe having a hairpin structure and having a sequence complementary to the first probe.

29. The method of claim 28, wherein particles of the hydrogel comprise at least one material selected from the group consisting of natural polymer, acrylic monomer or polymer, polyacrylamide-based monomer or polymer, phosphatidyl choline, hyaluronic acid-based monomer or polymer, carboxymethyl cellulose, alginate, chitosan, poly(e-caprolactone), poly(lactic acid), poly(glycolic acid), polyethylene glycol, hydroxyapatite, tricalcium phosphate, and mixtures thereof.

30. The method of claim 28, wherein the lipid constituting the liposome is any one or more selected from the group consisting of phospholipids, glycolipids, sterols, and cationic lipids.

31. The method of claim 28, wherein the reporter conjugated to the 5′ end is any one selected from the group consisting of ALEX-350, FAM, VIC, TET, CAL Fluor® Gold 540, JOE, HEX, CAL Fluor Orange 560, TAMRA, CAL Fluor Red 590, ROX, CAL Fluor Red 610, TEXAS RED, CAL Fluor Red 635, Quasar 670, CY3, CY5, CY5.5, and Quasar 705, and
the quencher conjugated to the 3′ end is any one selected from the group consisting of DABCYL, BHQ, ECLIPSE, and TAMRA.

32. The method of claim 28, wherein the method further comprises treating the liposome with a surfactant to degrade the membrane of the liposome.

33. The method of claim 28, wherein the sample is mixed in a buffer solution containing a surfactant.

34. The method of claim 33, wherein the surfactant degrades the membrane of the liposome to initiate the reaction of the sample with the hydrogel.

35. The method of claim 33, wherein the surfactant is one selected from the group consist of cetyl trimethylammonium bromide, hexadecyl trimethyl ammonium bromide, dodecyl betaine, dodecyl dimethylamine oxide, 3-(N,N-dimethylpalmitylammonio)propane sulfonate), Tween 20, Tween 80, Triton-X-100, polyethylene glycol monooleyl ether, triethylene glycol monododecyl ether, octyl glucoside, and N-nonanoyl-N-methylglucamine.

36. The method of claim 28, wherein the first probe comprises a sequence complementary to any one selected from the group consisting of mmu-miR-1187, mmu-miR-1306-3p, mmu-miR-7038-3p, mmu-miR-5113, mmu-miR-669n, mmu-miR-669c-5p, mmu-miR-365-2-5p, mmu-miR-3095-3p, mmu-miR-365-1-5p, mmu-miR-1931, mmu-miR-1306-5p, mmu-miR-7001-5p, mmu-miR-23a-5p, mmu-miR-574-5p, mmu-miR-3061-5p, mmu-miR-8117, mmu-miR-15a-3p, mmu-miR-665-5p, mmu-miR-669m-5p, mmu-miR-466m-5p, mmu-miR-668-5p, mmu-miR-6997-5p, mmu-miR-7684-5p, hsa-miR-1306-3p, hsa-miR-365b-5p, hsa-miR-365a-5p, hsa-miR-1306-5p, hsa-miR-23a-5p, hsa-miR-574-5p, hsa-miR-15a-3p, hsa-miR-665, and hsa-miR-668-5p.

37. A microfluidic chip for detecting microRNAs (miRNAs) comprising:
an inlet;
an outlet;
a microtubule passage connecting the inlet to the outlet; and
sensing parts of the outlet configured to be branched from the microtubule passage,
wherein the sensing part comprises a hydrogel,
wherein the hydrogel comprises a liposome comprising a first probe having a hairpin structure in which a reporter is conjugated to the 5′ end and a quencher is conjugated to the 3′ end and having a sequence complementary to a target miRNA; and a liposome comprising a second probe having a hairpin structure and having a sequence complementary to the first probe.

38. The microfluidic chip of claim 37, wherein the microtubule passage connecting the inlet to the outlet is connected so that a sample, a surfactant, or both are movable from the inlet to the outlet.

39. The microfluidic chip of claim 37, wherein the microfluidic chip includes two sensing parts of the outlet configured to be branched.


SEQUENCE LISTING (37 sequences)

SEQ ID NO: 1 - RNA, 23 nt, Artificial Sequence
  mmu-miR-1187
uauguguguguguauguguguaa

SEQ ID NO: 2 - RNA, 21 nt, Artificial Sequence
  mmu-miR-1306-3p
acguuggcucugguggugaug

SEQ ID NO: 3 - RNA, 22 nt, Artificial Sequence
  mmu-miR-7038-3p
cacugcuccugccuucuuacag

SEQ ID NO: 4 - RNA, 23 nt, Artificial Sequence
  mmu-miR-5113
acagaggaggagagagauccugu

SEQ ID NO: 5 - RNA, 20 nt, Artificial Sequence
  mmu-miR-669n
auuuguguguggaugugugu

SEQ ID NO: 6 - RNA, 22 nt, Artificial Sequence
  mmu-miR-669c-5p
auaguuguguguggaugugugu

SEQ ID NO: 7 - RNA, 23 nt, Artificial Sequence
  mmu-miR-365-2-5p
agggacuuucaggggcagcugug

SEQ ID NO: 8 - RNA, 22 nt, Artificial Sequence
  mmu-miR-3095-3p
aagcuuucucaucugugacacu

SEQ ID NO: 9 - RNA, 23 nt, Artificial Sequence
  mmu-miR-365-1-5p
agggacuuuugggggcagaugug

SEQ ID NO: 10 - RNA, 22 nt, Artificial Sequence
  mmu-miR-1931
augcaagggcuggugcgauggc

SEQ ID NO: 11 - RNA, 23 nt, Artificial Sequence
  mmu-miR-1306-5p
caccaccuccccugcaaacgucc

SEQ ID NO: 12 - RNA, 23 nt, Artificial Sequence
  mmu-miR-7001-5p
aggcagggugugagcgugagcau

SEQ ID NO: 13 - RNA, 22 nt, Artificial Sequence
  mmu-miR-23a-5p
gggguuccuggggaugggauuu

SEQ ID NO: 14 - RNA, 23 nt, Artificial Sequence
  mmu-miR-574-5p
ugagugugugugugugagugugu

SEQ ID NO: 15 - RNA, 22 nt, Artificial Sequence
  mmu-miR-3061-5p
cagugggccgugaaagguagcc

SEQ ID NO: 16 - RNA, 21 nt, Artificial Sequence
  mmu-miR-8117
gcucguguggaacagaagggg

SEQ ID NO: 17 - RNA, 22 nt, Artificial Sequence
  mmu-miR-15a-3p
caggccauacugugcugccuca

SEQ ID NO: 18 - RNA, 26 nt, Artificial Sequence
  mmu-miR-665-5p
aggggccucugccucuauccaggauu

SEQ ID NO: 19 - RNA, 23 nt, Artificial Sequence
  mmu-miR-669m-5p
ugugugcaugugcauguguguau

SEQ ID NO: 20 - RNA, 23 nt, Artificial Sequence
  mmu-miR-466m-5p
ugugugcaugugcauguguguau

SEQ ID NO: 21 - RNA, 24 nt, Artificial Sequence
  mmu-miR-668-5p
guaagugugccucgggugagcaug

SEQ ID NO: 22 - RNA, 22 nt, Artificial Sequence
  mmu-miR-6997-5p
uaacaggcuggagaggugcaga

SEQ ID NO: 23 - RNA, 21 nt, Artificial Sequence
  mmu-miR-7684-5p
ucugggaagccugggcagcag

SEQ ID NO: 24 - RNA, 18 nt, Artificial Sequence
  hsa-miR-1306-3p
acguuggcucugguggug

SEQ ID NO: 25 - RNA, 22 nt, Artificial Sequence
  hsa-miR-365b-5p
agggacuuucaggggcagcugu

SEQ ID NO: 26 - RNA, 23 nt, Artificial Sequence
  hsa-miR-365a-5p
agggacuuuugggggcagaugug

SEQ ID NO: 27 - RNA, 22 nt, Artificial Sequence
  hsa-miR-1306-5p
ccaccuccccugcaaacgucca

SEQ ID NO: 28 - RNA, 22 nt, Artificial Sequence
  hsa-miR-23a-5p
gggguuccuggggaugggauuu

SEQ ID NO: 29 - RNA, 23 nt, Artificial Sequence
  hsa-miR-574-5p
ugagugugugugugugagugugu

SEQ ID NO: 30 - RNA, 22 nt, Artificial Sequence
  hsa-miR-15a-3p
caggccauauugugcugccuca

SEQ ID NO: 31 - RNA, 20 nt, Artificial Sequence
  hsa-miR-665
accaggaggcugaggccccu

SEQ ID NO: 32 - RNA, 19 nt, Artificial Sequence
  hsa-miR-668-5p
ugcgccucgggugagcaug

SEQ ID NO: 33 - DNA, 54 bp, Artificial Sequence
  H1
tgtgtgtgtgagtgtgggatcgaaagtgtgtgcacactcacacacacacactca

SEQ ID NO: 34 - DNA, 58 bp, Artificial Sequence
  H2
tgtgggatcgaaagtgtgtgtgtgtgagtgtgcacacactttcgatcccacactcaca

SEQ ID NO: 35 - DNA, 23 bp, Artificial Sequence
  Target-574
tgagtgtgtgtgtgtgagtgtgt

SEQ ID NO: 36 - DNA, 23 bp, Artificial Sequence
  1MS-574
tgagtgtgggtgtgtgagtgtgt

SEQ ID NO: 37 - DNA, 23 bp, Artificial Sequence
  2MS-574
tgagtctgggtgtgtgagtgtgt